(19) United States
(12) Patent Application Publication
(10) Pub. No.: US 2009/0133133 A1
(43) Pub. Date: May 21, 2009

(54) Transgenic Mice for Bioassay of Prions from Deer and Elk with Chronic Wasting Disease

(75) Inventor: Glenn C. Telling, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/251,183

(22) Filed: Oct. 14, 2008

Related U.S. Application Data
(62) Division of application No. 11/598,086, filed on Nov. 13, 2006, now abandoned.
(60) Provisional application No. 60/597,138, filed on Nov. 11, 2005.

(51) Int. Cl. G01N 33/53; A01K 67/027; C12N 15/31; C12N 15/11

(52) U.S. Cl. 800/3; 800/14; 800/15; 800/16; 800/17; 800/21; 536/23.7

(57) ABSTRACT

The invention relates to the use of transgenic constructs to produce animal models for the study of chronic wasting disease.

### STATEMENT REGARDING FEDERALLY SPONSORED RESEARCH

This invention was funded in part by Grant V180003 from the U.S. Department of Defense, therefore the government may have certain rights in the invention.

### TECHNICAL FIELD

The invention relates to the use of transgenic constructs to produce animal models for the study of chronic wasting disease.

### BACKGROUND

Chronic Wasting Disease (CWD) is a transmissible neurological disease of deer and elk (and possibly other members of the deer family) that is believed to be caused by prion infection that leads to the production of small lesions in the brains of infected animals. Symptoms include loss of body condition, behavioral abnormalities, and eventually can cause death. CWD is classified as a transmissible spongiform encephalopathy (TSE), and is related to other prion-associated diseases, such as mad cow disease in cattle and scrapie in sheep and goats.

CWD is of increasing concern in the United States, as CWD can reduce the growth and size of wild deer and elk populations in areas where the prevalence is high. The disease was previously thought to be limited in the wild to a relatively small endemic area in northeastern Colorado, southeastern Wyoming and southwestern Nebraska. However, CWD has recently been found in several new areas across the United States. The disease also has been diagnosed in commercial game farms in several U.S. states. Three members of the deer family are known to be especially susceptible to CWD—elk, mule deer and white-tailed deer. Susceptibility of other members of the deer family (i.e., cervids) is possible.

Although related, CWD is distinctly different from “mad cow disease”. While there is presently no evidence that CWD poses a risk for humans, health officials recommend that human exposure to the CWD infectious agent is avoided. Hunters are encouraged not to consume meat from animals known to be infected.

In response to the potential risk of transmission to humans, wildlife managers have implemented many precautionary programs. However, surveillance programs are expensive and draw resources from other wildlife management needs. One option for managing CWD in wild populations is to reduce the density of animals in the infected area to slow the transmission of the disease. When CWD is detected in a captive cervid facility, generally that facility is quarantined and all captive cervids in that facility are killed.

Accordingly, there is a need to develop the systems and methods to study CWD in the cervid family to have further knowledge regarding the etiology of the disease, and to assist in the management of transmission to noninfected cervids and other animals.

### SUMMARY

The invention relates to a genetically modified non-human animal (e.g., rat or mouse) for studying chronic wasting disease (CWD), said genetically modified non-human animal being transgenically altered to express cervid prion protein (CerPrP), wherein said non-human animal produces infected prions (PrP^{sc}) upon infection with prions. The invention further relates to a method for making said genetically modified non-human animal comprising the steps of constructing a construct containing an open reading frame of CerPrP, and transforming the non-human animal with the construct. The invention further relates to a bioassay for studying CWD, comprising intracerebrally inoculating a transgenic non-human animal that expresses CerPrP with a biological sample of tissue, body fluid, blood or secretion from a non-human suspected of suffering from CWD, and assaying for signs of CWD. The invention further relates to a method for screening for therapeutic agents useful for treating CWD, comprising inoculating a potential agent for treating CWD, and determining the effects of the potential therapeutic agent on the development of CWD using a transgenic animal of the invention or cells obtained therefrom. The invention further relates to a method for studying the molecular and biochemical events associated with chronic wasting disease, comprising inoculating transgenic CerPrP non-human animals, inoculating wild-type animals of the same species, and comparing signs of CWD from said animals. In one aspect, the non-human transgenic animal is a mouse.

### BRIEF DESCRIPTION OF THE DRAWINGS

FIG. 1A-D depicts the post-infection severe vacuolation of hippocampus (A and B) and amyloid plaque formation (C and D) in Tg(CerPrP)I536^{+/-} mice inoculated with CWD prions.

FIG. 2A-B depicts western blots in brains from Tg(CerPrP)I536^{+/-} mice inoculated with CWD prions.

FIG. 3 depicts the regional distribution of PrP^{Sc} in the brains of Tg(CerPrP)I536^{+/-} mice inoculated with CWD prions.

FIG. 4 depicts a schematic of pSPOXII.PrP→neo. The relative locations of functional regions of a generic PrP expression construct are shown.

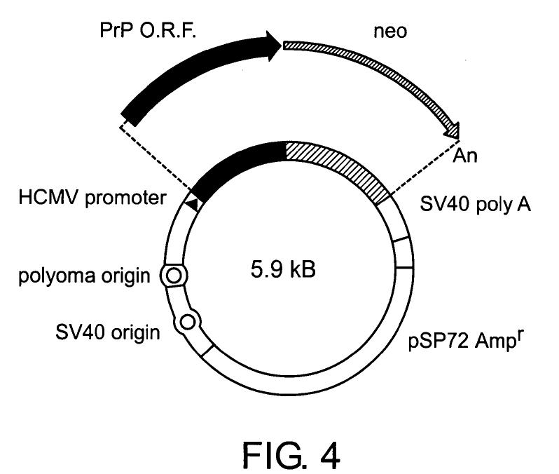

FIG. 5A-B depicts transgene expression levels in mice using two different expression vectors: a plasmid-based vector (pMo53) and cosmid vector cosSHa.tet.

FIG. 6A-B depicts transgenic models of human, bovine and ovine prion diseases as a means of assessing susceptibility of humans and livestock to CWD infection.

### DETAILED DESCRIPTION

The invention relates to the use of transgenic constructs to produce animal models and the animals they produced for the study of chronic wasting disease.

“Transgenic animal” refers to a non-human animal into which exogenous DNA has been introduced while the animal is still in its embryonic stage. In most cases, the transgenic approach aims at specific modifications of the genome, e.g., by introducing whole transcriptional units into the genome, or by up- or down-regulating pre-existing cellular genes. The targeted character of certain of these procedures sets transgenic technologies apart from experimental methods in which random mutations are conferred to the germline, such as administration of chemical mutagens or treatment with ionizing solution.

The term “chimera,” “mosaic,” “chimeric animal” and the like, refers to a transgenic animal with a knockout, mutation, or recombinant construct, in some of its genome-containing cells.

The term “heterozygote,” “heterozygotic animal” and the like, refers to a transgenic animal with a disruption, mutation, or construct on one of a chromosome pair in all of its genome-containing cells.

The term “homozygote,” “homozygotic animal” and the like, refers to a transgenic animal with a disruption on both members of a chromosome pair in all of its genome-containing cells.

A “non-human animal” of the invention includes animals such as rodents, non-human primates, sheep, dog, amphibians, reptiles, avian such as meat bred and egg laying chicken and turkey, ovine such as lamb, bovine such as beef cattle and milk cows, piscine and porcine, and cervids, such as deer and elk.

It must be noted that as used herein and in the appended claims, the singular forms “a” or “an” or “the” include plural referents unless the context clearly dictates otherwise. Thus, for example, reference to “a protein” includes a plurality of such proteins and reference to “the antibody” includes reference to one or more antibodies and equivalents thereof known to those skilled in the art, and so forth.

Unless defined otherwise, all technical and scientific terms used herein have the same meaning as commonly understood to one of ordinary skill in the art to which this invention belongs.

Although the invention uses a typical non-human rodent animal (e.g., including rat and mouse) other animals can similarly be genetically modified using the methods and compositions of the invention.

Typically, the genome of the transgenic non-human animal comprises an inserted heterologous polynucleotide or one or more deletions in one or more exons of the genes and further comprises a heterologous selectable marker gene.

Techniques for obtaining the transgenic animals of the invention are well known in the art; the techniques for introducing foreign DNA sequences into the mammalian germ line were originally developed in mice. One route of introducing foreign DNA into a germ line entails the direct microinjection of linear DNA molecules into a pronucleus of a fertilized one-cell egg. Microinjected eggs are subsequently transferred into the oviducts of pseudopregnant foster mothers and allowed to develop. About 25% of the progeny mice inherit one or more copies of the micro-injected DNA. Currently, the most frequently used techniques for generating chimeric and transgenic animals are based on genetically altered embryonic stem cells or embryonic germ cells. Techniques suitable for obtaining transgenic animals have been amply described. A suitable technique for obtaining completely ES cell derived transgenic non-human animals is described in WO 98/06834.

Transgenic animals of the invention can be obtained by standard genetic manipulation methods as described herein, typically by using ES cells. Thus, the invention relates to a method for producing a transgenic non-human animal comprising (i) providing an embryonic stem (ES) cell from the relevant animal species that is PrP ⁻¹⁻ or comprising a first intact PrP gene; (ii) providing a construct capable of inserting a homologous PrP (e.g., CerPrP) or disrupting the intact PrP gene and inserting a heterologous gene; (iii) introducing the targeting construct into the ES cells under conditions where the heterologous PrP of the construct undergoes homologous recombination with the genome of the non-human animal; (iv) introducing the ES cells carrying a heterologous PrP (e.g., CerPrP) gene into a blastocyst; (v) implanting the blastocyst into the uterus of pseudopregnant female; (vi) delivering animals from said females, identifying a mutant animal that carries the recombinant gene allele and (vii) selecting for transgenic animals and breeding them.

A “targeting construct” is a construct comprising sequences that can be inserted into the genome of a non-human animal, e.g., by homologous recombination. The targeting construct generally has a 5′ flanking region and a 3′ flanking region homologous to segments of the genome where the heterologous polynucleotide is to be inserted. These homologous segments surround a foreign DNA sequence (e.g., a mutant gene or new gene) to be inserted into the genome. For example, the foreign DNA may encode a selectable marker, such as an antibiotics resistance gene or mutant gene. Examples for suitable selectable markers are the neomycin resistance gene (NEO) and the hygromycin β-phosphotransferase gene. The 5′ flanking region and the 3′ flanking region are homologous to regions within the gene surrounding the portion of the gene to be replaced with the heterologous (e.g., mutant) DNA. DNA comprising the targeting construct and the native gene of interest are contacted under conditions that favor homologous recombination. For example, the construct and native gene sequence of interest can be used to transform embryonic stem (ES) cells, in which they can subsequently undergo homologous recombination.

Thus, a targeting construct refers to a polynucleotide that can be used to decrease or suppress expression of a protein encoded by endogenous DNA sequences in a cell or which encodes a mutant or heterologous protein resulting in expression of the protein. Alternatively, a number of termination codons can be added to the native polynucleotide to cause early termination of the protein or an intron junction can be inactivated. In a typical construct, some portion of the polynucleotide comprises a selectable marker (such as the neo gene), upstream or downstream relative to a portion of the CerPrP polynucleotide and where neo refers to a neomycin resistance gene.

A targeting construct refers to a uniquely configured polynucleotide which is introduced into a stem cell line and allowed to recombine with the genome at the chromosomal locus of the gene of interest. Typically, a given construct is specific for a given gene to be targeted. Nonetheless, many common elements exist among these constructs and these elements are known in the art. A typical targeting construct contains not less than about 0.5 kb nor more than about 10.0 kb from both the 5′ and the 3′ ends of the genomic locus which encodes the gene locus to be targeted. These two fragments are separated by an intervening fragment comprising the mutant gene or heterologous gene to be inserted, and may also include a polynucleotide encoding a positive selectable marker, such as the neomycin resistance gene (neo^{R}). The resulting construct comprises a polynucleotide from the extreme 5′ end of the genomic locus being targeted linked to a polynucleotide encoding a heterologous PrP (e.g., CerPrP) and may also include a positive selectable marker which is in turn linked to a nucleic acid from the extreme 3′ end of the genomic locus of interest. When the resulting construct recombines homologously with the chromosome at this locus, it results in the insertion of the PrP polynucleotide into the genomic locus. A stem cell in which such a homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Proper homologous recombination can be confirmed by Southern blot analysis of restriction endonuclease digested DNA using, as a probe, a segment of the construct. Since the native genome segment will exhibit a restriction pattern different from that of the inserted construct, the presence of an inserted construct can be determined from the size of the restriction fragments that hybridize to the probe.

In an animal obtained by the methods above, the extent of the contribution of the ES cells that contain the heterologous PrP (e.g., CerPrP) gene to the somatic tissues of the transgenic animal can be determined visually by choosing animal strains for the source of the ES cells and blastocyst that have different coat colors.

In one embodiment, the transgenic animals of the invention are mice. In other embodiments of this invention, the animals are rodents, guinea pigs, rabbits, non-human primates, sheep, dog, cow, amphibians, reptiles, avian such as meat bred and egg laying chicken and turkey, ovine such as lamb, bovine such as beef cattle and milk cows, piscine and porcine. The transgenic animals can be used for a variety of purposes, e.g., to identify therapeutics agents for treating CWD.

The transgenic animals can typically contain a transgene, such as reporter gene, under the control of a PrP (e.g., CerPrP) promoter or fragment thereof. Methods for obtaining transgenic and knockout non-human animals are known in the art. Knock out mice are generated by homologous integration of a “targeting construct” construct into a mouse embryonic stem cell chromosome which encodes a gene to be knocked out. In one embodiment, gene targeting, which is a method of using homologous recombination to modify an animal's genome, can be used to introduce changes into cultured embryonic stem cells. By targeting an PrP gene of interest in ES cells, these changes can be introduced into the germlines of animals to generate chimeras. The gene targeting procedure is accomplished by introducing into tissue culture cells a DNA targeting construct that includes a segment homologous to a target PrP locus, and which also includes an intended sequence modification to the PrP genomic sequence (e.g., insertion, deletion, point mutation). The treated cells are then screened for accurate targeting to identify and isolate those which have been properly targeted. In one aspect, PrP ⁻¹⁻ animals are used to generate transgenic organisms that comprise a heterologous PrP (e.g., a CerPrP). In another aspect, animals with wild-type PrP are used such that the wild-type PrP is disrupted by a heterologous PrP.

Generally, the embryonic stem cells (ES cells) used to produce the transgenic animals will be of the same species as the transgenic animal to be generated. Thus for example, mouse embryonic stem cells will usually be used for generation of transgenic mice.

Embryonic stem cells are generated and maintained using methods well known to the skilled artisan such as those described by Doetschman et al. (1985) J. Embryol. Exp. Mol. Biol. 87:27-45). Any line of ES cells can be used, however, the line chosen is typically selected for the ability of the cells to integrate into and become part of the germ line of a developing embryo so as to create germ line transmission of the transgenic construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934). Still another ES cell line is the WW6 cell line (Ioffe et al. (1995) PNAS 92:7357-7361). The cells are cultured and prepared for transgenic construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. (1986)).

As mentioned above, the homologous recombination of the above described constructs is sometimes rare and such a construct can insert nonhomologously into a random region of the genome where it has no effect on the gene which has been targeted for deletion, and where it can potentially recombine so as to disrupt another gene which was otherwise not intended to be altered. Such non-homologous recombination events can be selected against by modifying the above-mentioned targeting constructs so that they are flanked by negative selectable markers at either end (particularly through the use of two allelic variants of the thymidine kinase gene, the polypeptide product of which can be selected against in expressing cell lines in an appropriate tissue culture medium known in the art—i.e. one containing a drug such as 5-bromodeoxyuridine). Non-homologous recombination between the resulting targeting construct comprising the negative selectable marker and the genome will usually result in the stable integration of one or both of these negative selectable marker genes and hence cells which have undergone non-homologous recombination can be selected against by growth in the appropriate selective media (e.g., media containing a drug such as 5-bromodeoxyuridine for example). Simultaneous selection for the positive selectable marker and against the negative selectable marker will result in a vast enrichment for clones in which the construct has recombined homologously at the locus of the gene intended to be mutated. The presence of the predicted chromosomal alteration at the targeted gene locus in the resulting transgenic stem cell line can be confirmed by means of Southern blot analytical techniques which are well known to those familiar in the art. Alternatively, PCR can be used.

Each targeting construct to be inserted into the cell is linearized. Linearization is accomplished by digesting the DNA with a suitable restriction endonuclease selected to cut only within the construct sequence and not the 5′ or 3′ homologous regions or the selectable marker region.

For insertion, the targeting construct is added to the ES cells under appropriate conditions for the insertion method chosen, as is known to the skilled artisan. For example, if the ES cells are to be electroporated, the ES cells and targeting construct are exposed to an electric pulse using an electroporation machine and following the manufacturer's guidelines for use. After electroporation, the ES cells are typically allowed to recover under suitable incubation conditions. The cells are then screened for the presence of the targeting construct as explained herein. Where more than one construct is to be introduced into the ES cell, each targeting construct can be introduced simultaneously or one at a time.

After suitable ES cells containing the construct in the proper location have been identified by the selection techniques outlined above, the cells can be inserted into an embryo. Insertion may be accomplished in a variety of ways known to the skilled artisan, however the typical method is by microinjection. For microinjection, about 10-30 cells are collected into a micropipet and injected into embryos that are at the proper stage of development to permit integration of the foreign ES cell containing the recombination construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan.

While any embryo of the right stage of development is suitable for use, typical embryos are male. In mice, the typical embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the transgenic offspring are available. For example, Northern blots can be used to probe the mRNA for the presence or absence of transcripts encoding either the transgenic gene, the marker gene, or both. In addition, Western blots can be used to assess the level of expression of the PrP gene (e.g., CerPrP) in various tissues of the offspring by probing the Western blot with an antibody against the particular PrP, or an antibody against the marker gene product. Finally, in situ analysis (such as fixing the cells and labeling with antibody) and/or FACS (fluorescence activated cell sorting) analysis of various cells from the offspring can be conducted using suitable antibodies to look for the presence or absence of the construct's gene product(s).

In another aspect, a transgenic animal can be obtained by introducing into a single stage embryo a targeting construct of the invention. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has an advantage in that in most cases the injected DNA (e.g., the injected construct) will be incorporated into the host gene before the first cleavage (Brinster et al. (1985) PNAS 82:4438-4442). As a consequence, all cells of the transgenic animal will carry the incorporated nucleic acids of the targeting construct. This will in general also be reflected in the efficient transmission to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus. In some species such as mice, the male pronucleus is typically used. Typically the exogenous genetic material be added to the male DNA complement of the zygote prior to its being processed by the ovum nucleus or the zygote female pronucleus. It is thought that the ovum nucleus or female pronucleus release molecules which may affect the male DNA complement, perhaps by replacing the protamines of the male DNA with histones, thereby facilitating the combination of the female and male DNA complements to form the diploid zygote.

Thus, the exogenous genetic material is typically added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the a exogenous nucleic acid (e.g., a targeting construct) into the embryo may be accomplished by any means known in the art such as, for example, microinjection, electroporation, or lipofection. Following introduction of the exogenous nucleic acid into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is used. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of a transgene (e.g., the exogenous genetic material or targeting constructs) which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of a targeting construct, in order to insure that one copy is functional.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of an exogenous polynucleotide (e.g., that of a targeting construct) by any suitable method as described herein. Alternative or additional methods include biochemical assays such as enzyme and/or immunological assays, histological stains for particular marker or enzyme activities, flow cytometric analysis, and the like.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different knockout, or both. Alternatively, the partner may be a parental line. Where in vitro fertilization is used, the fertilized embryo may be implanted into a surrogate host or incubated in vitro, or both. Using either method, the progeny may be evaluated using methods described above, or other appropriate methods.

Retroviral infection can also be used to introduce a targeting construct into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the targeting vector is typically a replication-defective retrovirus carrying the exogenous nucleic acid (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the targeting construct (e.g., the exogenous nucleic acids) since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

### Targeting Constructs

The invention provides a targeting construct useful for generating transgenic non-human animals of the invention, such as rodents, guinea pigs, rabbits, non-human primates, sheep, dog, cow, amphibians, reptiles, avian such as meat bred and egg laying chicken and turkey, ovine such as lamb, bovine such as beef cattle and milk cows, piscine and porcine. The targeting construct comprises a polynucleotide of the open reading frame cassette of the CerPrP S2 allele (GenBank accession no. AF009180). The CosSHa.Tet cosmid construct contains a 49 kb DNA fragment encompassing the Syrian hamster PrP gene (Scott, M. R. et al. (1992) Chimeric prion protein expression in cultured cells and transgenic mice, Protein Sci. 1:986-997) and has been used to produce numerous transgenic models of prion diseases (Telling, G. C. (2000) Prion protein genes and prion diseases: Studies in transgenic mice, Neuropathology and Applied Neurobiology 26:209-220) including mice in which the species varies to Syrian hamster, human and bovine prions are eliminated (Asante, E. A. et al. (2002) BSE prions propagate as either variant CID-like or sporadic CJD-like prion strains in transgenic mice expressing human prion protein, Embo J 21:6358-66; Prusiner, S. B. et al. (1990) Transgenetic studies implicate interactions between homologous PrP isoforms in scrapie prion replication, Cell 63:673-686; Scott, N I. et al. (1989) Transgenic mice expressing hamster prion protein produce species-specific scrapie infectivity and amyloid plaques, Cell 59:847-857; Scott, M. R. et al. (1997) Identification of a prion protein epitope modulating transmission of bovine spongiform encephalopathy prions to transgenic mice, Proc. Natl. Acad. Sci. USA 94:14279-14284; Telling, G. C. et al. (1994) Transmission of Creutzfeldt-Jakob disease from humans to transgenic mice expressing chimeric human-mouse prion protein, Proc Natl Acad Sci USA, 91:9936-40; and Telling, G. C et al. (1995) Prion propagation in mice expressing human and chimeric PrP transgenes implicates the interaction of cellular PrP with another protein, Cell 83:79-90).

In one aspect, the ORF of the CerPrP S2 allele (Genbank # AF009180) is cloned into a targeting construct. In another aspect, the ORF cassette of the CerPrP S2 allele (Genbank # AF009180) can be released following digestion with SalI and XhoI, and ligated to the SalI-cut cosSHa.Tet cosmid construct, resulting in the production of a CerPrP-encoding construct. Using the construct described above, which carries the CerPrP gene, animals (e.g., mice) can be engineered to render them capable of overexpressing CerPrP. Using the methods and targeting construct herein, a mouse model, Tg(CerPrP) mouse, were generated that overexpress CerPrP.

### Using Tg(CerPrP) Mice

The Tg(CerPrP) transgenic non-human animals (e.g., mice) of the invention are useful in detecting the presence of infectious prions in brain isolates from animals suspected of having prion associated disease (e.g., cervids suspected of having CWD). For example, such transgenic non-human animals can be intracellularly inoculated with brain isolates and monitored for signs of prion disease. Prion-associated diseases and disorders include all forms of spongiform encephalopathies. Characteristics of the spongiform encephalopathies include the appearance of the brain with large vacuoles in the cortex and cerebellum. Specific examples of prion-associated diseases and disorders include, but are not limited to, Scrapie in sheep, TME (transmissible mink encephalopathy) in mink, CWD (chronic wasting disease) in muledeer and elk, BSE (bovine spongiform encephalopathy) in bovines and particularly cows, CJD (Creutzfeld-Jacob Disease) in humans, GSS (Gerstmann-Straussler-Scheinker syndrome) in humans, FFI (Fatal familial Insomnia) in humans, Kuru in humans, and Alpers Syndrome in humans.

For example, the simulation of CWD in deer and elk following transmission to Tg(CerPrP) mice as taught by the invention will facilitate CWD research. Tg(CerPrP) mice are useful in the study of the biology of CWD prions and CWD pathogenesis. There is currently no quantitative information available regarding the infectivity of any CWD prion preparations, and the Tg(CerPrP) mice of the invention provide a reliable experimental host in which to bioassay CWD prions. Using Tg(CerPrP) mice investigations can be conducted of CWD prion strain prevalence in captive and wild populations of mule deer, white tailed deer and Rocky Mountain elk and to assess the effect of cervid PrP polymorphisms on CWD susceptibility (Johnson, C. et al. (2003) Prion protein gene heterogeneity in free-ranging white-tailed deer within the chronic wasting disease affected region of Wisconsin, J Wildl Dis 39:576-81; and O'Rourke, K. I. et al. (1999) PrP genotypes of captive and free-ranging Rocky Mountain elk (Cervus elaphus nelsoni) with chronic wasting disease, J Gen Virol 80 (Pt 10):2765-9). These models additionally facilitate study of the origins and mode of transmission of CWD. Efficient horizontal rather than maternal transmission has been shown to be important in sustaining CWD epidemics (Miller, M. W. et al. (2003) Prion disease: horizontal prion transmission in mule deer, Nature 425:35-6). The most plausible natural route(s) of CWD transmission are via ingestion of forage or water contaminated by secretions, excretions, or other sources of agent, for example carcasses (Miller, M. et al. (2004) Environmental sources of prion transmission in mule deer, Emerging Infectious Diseases 10). Using CWD susceptible Tg(CerPrP) mice, bioassay can be conducted for CWD prions in blood and other tissues, body fluids and secretions of deer and elk that will provide information regarding the mode of transmission of CWD, which may lead to better disease control in wild cervids.

The invention further provides a method for screening for therapeutic agents useful for treating prion-associated disease. The method comprises inoculating a potential agent for treating prion-associated disease and then determining the effects of the potential therapeutic agent on the development of prion-associated disease. Examples of prion-associated diseases include Scrapie in sheep, TME (transmissible mink encephalopathy) in mink, CWD (chronic wasting disease) in muledeer and elk, BSE (bovine spongiform encephalopathy) in bovines and particularly cows, CJD (Creutzfeld-Jacob Disease) in humans, GSS (Gerstmann-Straussler-Scheinker syndrome) in humans, FFI (Fatal familial Insomnia) in humans, Kuru in humans, and Alpers Syndrome in humans.

The invention further provides a method for studying the molecular and biochemical events associated with prion disease. The method comprises a) inoculating transgenic CerPrP mice; b) inoculating wild-type mice; and then comparing signs of prion disease from the mice in step a. to the mice in step b.

### EXAMPLES

### Example 1

### Preparation of Tg(CerPrP) Mice

Transgenic mice were generated, expressing cervid prion protein (PrP), to produce a transgenic system simulating chronic wasting disease (CWD) in deer and elk. The mice were referred to as Tg(CerPrP) mice. To produce the Tg(CerPrP) mice, the open reading frame cassette of the CerPrP S2 allele (Genbank #AF009180) was released from plasmid sequences following digestion with SalI and XhoI and purified ORF fragments were ligated to the SalI-cut cosSHa.Tet cosmid construct.

To increase CerPrP expression in Tg mice, the CerPrP 52 allele plasmid nucleotide sequence was modified by site-directed mutagenesis immediately upstream of the initiating ATG to produce a consensus Kozak translation initiation sequence.

Two founders were generated by microinjection of fertilized embryos from Prnp^{0/0} knockout mice on an FVB/N background (FVB/Prnp^{0/0}). Brain PrP expression was estimated by comparing serially diluted brain extracts of Fl Tg mice and wild type mice followed by both immuno-dot blotting and western blotting using the monoclonal antibody 6H4 (Prionics AG, Schlieren). By this approach, the levels of CerPrP expression in brain extracts of Tg(CerPrP)1536^{+/-} and Tg(CerPrP)1534^{+/-} mice, both hemizygous for the transgene array, were estimated to be 5- and 2-fold higher, respectively, than the level of wild type PrP expression in FVB mice.

### Example 2

### Production of CWD in Tg(CerPrP) Mice Using CWD Positive Brain Isolates

Groups of Tg(CerPrP)1536^{+/-} mice were intracerebrally inoculated with 30 μl of 1% homogenate prepared in phosphate buffered saline (PBS) of a pooled collection of infected brains from CWD affected mule deer held captive at the Colorado Division of Wildlife, Wildlife Research Center. The transmission of CWD isolates from individual captive mule deer and elk in Tg(CerPrP)I536^{+/-} mice was also compared. Samples Dl0 and Db99 refer to captive mule deer does that developed CWD at the Colorado Division of Wildlife Research Facility, and sample 7378 refers to an adult female captive elk with natural clinical CWD from the Wyoming Game and Fish Department's Sybille Wildlife Research Unit, Wheatland, Wyo. Inoculated Tg(CerPrP) 1536^{+/-} mice developed signs of prion disease between 220 and 270 days after inoculation, and the average incubation period produced by all three CWD isolates and the CWD pool were similar (see Table 1). The CWD Pool inoculum also produced disease in the inoculated Tg(CerPrP) 1534^{+/-} mice between 261 and 273d (see Table 1). The neurological signs that accompanied prion disease in sick Tg mice were remarkably consistent and included truncal ataxia and slowed movement, increased tone of the tail, dorsal kyphosis, head bobbing or tilting and roughened coat. Tg(CerPrP)1536^{+/-} control mice inoculated with PBS or mouse-adapted RML scrapie prions did not show signs of neurological dysfunction ˜320 and 350 days post-inoculation, respectively. Wild type mice inoculated with the CWD pool also failed to develop signs of neurological dysfunction ˜600 days post-inoculation.

TABLE 1
Transmission of CWD prions to Tg(CerPrP) mice

| | | | Mean ± SE incubation | |
|---|---|---|---|---|
| Inoculum | Species | Recipient | time in days^{a} | |
| CWD pool | Mule deer | Tg(CerPrP)1536^{+/-} | 264 ± 3 | (7/7) |
| Db99 | Mule deer | Tg(CerPrP)1536^{+/-} | 259 ± 4 | (7/7) |
| D10 | Mule deer | Tg(CerPrP)1536^{+/-} | 225 ± 1 | (8/8) |
| 7378 | Elk | Tg(CerPrP)1536^{+/-} | 235 ± 2 | (8/8) |
| RML | Mouse-adapted | Tg(CerPrP)1536^{+/-} | >385 | (0/8) |
| | scrapie | | | |
| PBS | | Tg(CerPrP)1536^{+/-} | >360 | (0/8) |
| D10 | Mule deer | Tg(CerPrP)1536^{+/-} | 160 ± 3 | (7/7) |
| CWD pool | Mule deer | Tg(CerPrP)1534^{+/-} | 268 ± 2 | (10/10) |
| PBS | | Tg(CerPrP)1534^{+/-} | >300 | (0/6) |
| CWD pool | Mule deer | Wild-type mice | >596 | (0/7) |

^{a}The number of mice developing clinical signs of prion disease divided by the original number of inoculated mice is shown in parentheses.

### Example 3

### Histopathological Studies of CWD Infected Tg(CerPrP) Mice

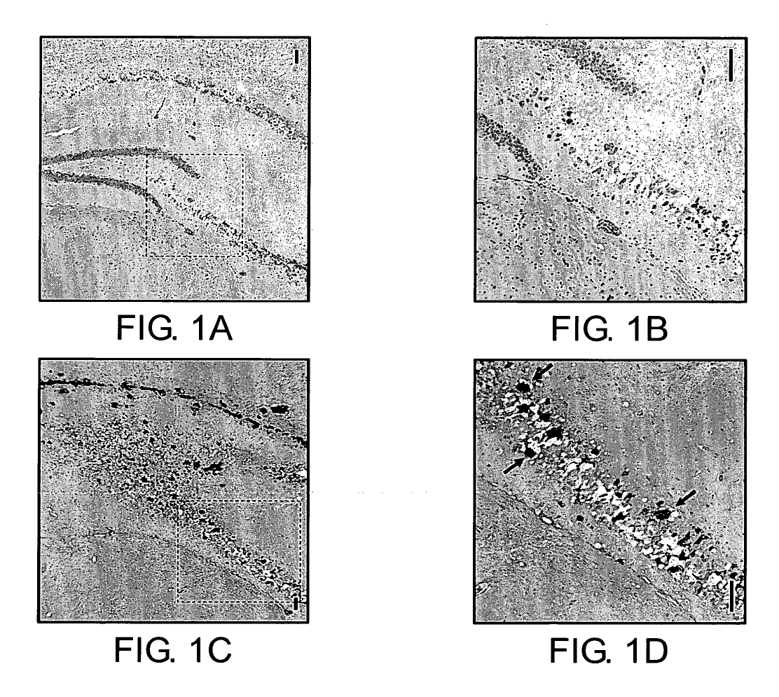

FIG. 1 shows sections from the brains of the study animals. The brains of sick animals from each study group were dissected rapidly after sacrifice and immersion fixed in 10% buffered paraformaldehyde. Tissue was embedded in paraffin and sections prepared and stained with hematoxylin and eosin (H&E) for evaluation of spongiform degeneration. A and B: H&E staining of sections through the hippocampus of Tg(CerPrP)I536^{+/-} mice inoculated with brain tissue from CWD-affected mule deer D10 showing spongiform degeneration. B is a magnification of the area indicated in A. Note shrunken, scalloped neuronal nuclei adjacent to foci of spongiform change. C and D show the immunohistochemistry of an adjacent section from the same inoculated Tg(CerPrP)I536^{+/-} mouse showing amyloid plaque deposits. D is a magnification of the area indicated in C. Note large immunoreactive plaques bordered by vacuoles indicated by arrows. Slides were deparaffinized, and hydrated followed by immersion in 88% formic acid solution, treatment with 25 mg/ml proteinase-K solution at 26° C. for 10 minutes, followed by autoclaving for 20 minutes at 121° C. in Tris buffered solution. Tissue preparations were stained using anti-PrP polyclonal antibody R505 (Garssen, G. J et al. (2000) Applicability of three anti-PrP peptide sera including staining of tonsils and brainstem of sheep with scrapie, Microsc Res Tech 50:32-9), followed by anti-rabbit IgG biotinylated secondary antibody, streptavidin conjugated to alkaline phosphatase, and then developed with Fast Red A, Naphthol, and Fast Red B chromagen. Hematoxylin was used as counterstain. Bar=100 μm in all cases.

Histopathologic findings were similar for all four inocula and included multiple to coalescing foci of spongiform degeneration of the perikaryon and neuropil. Foci of degeneration were often severe with a central focus of pale eosinophilic reticulated material surrounded by vacuoles. Neurons adjacent to foci of spongiform change often had shrunken scalloped hyperchromatic nuclei. While spongiform change was widespread in the brain there was striking and severe vacuolation of the hippocampus (see FIGS. 1 A and B), piriform cortex and parenchyma adjacent to the ventricular and aqueduct system throughout the brain. In all brains, spongiform degeneration was present in many nuclei in the sub-cerebellar white matter and brainstem. Patchy foci of degeneration were often present in the middle lamina of the neocortex, within the granular layer of the cerebellar cortex and within the olfactory bulb. Amyloid plaque pathology, long recognized as a pathognomonic feature in cervids with CWD (Guiroy, D. C. et al. (1991) Immunolocalization of scrapie amyloid (PrP27-30) in chronic wasting disease of Rocky Mountain elk and hybrids of captive mule deer and white-tailed deer, Neurosci Left 126: 195-8; Guiroy, D. C. et al. (1991) Topographic distribution of scrapie amyloid-immunoreactive plaques in chronic wasting disease in captive mule deer, Acta Neuropathol (Berl) 81:475-8; and Williams, E. S. et al. (1993) Neuropathology of chronic wasting disease of mule deer (Odocoileus hemionus) and Elk (Cervus elaphus nelsoni), Vet Pathol 30:36-45) was reproduced in Tg mice (see FIGS. 1 C and D). All foci of spongiform change had strong positive immunostaining (see FIGS. 1 C and D), often with large central stained plaques partly bordered, or surrounded by, non-staining vacuoles. Such florid PrP plaque pathology has also been recognized as a neuropathological feature of CWD (Liberski, P. P. et al. (2001) Deposition patterns of disease-associated prion protein in captive mule deer brains with chronic wasting disease, Acta Neuropathol (Berl) 102:496-500). Sham inoculated mice analyzed in parallel had no histologic lesions or positive immunostaining, neither was immunostaining identified in CWD positive deer brain or CWD inoculated Tg mice when an irrelevant primary antibody was used and when no primary antibody was applied. Brain tissue from a CWD positive deer had excellent positive immunostaining with the protocol used.

### Example 4

### Biochemical Analysis of Prion Proteins in CWD Infected Tg(CerPrP) Mice

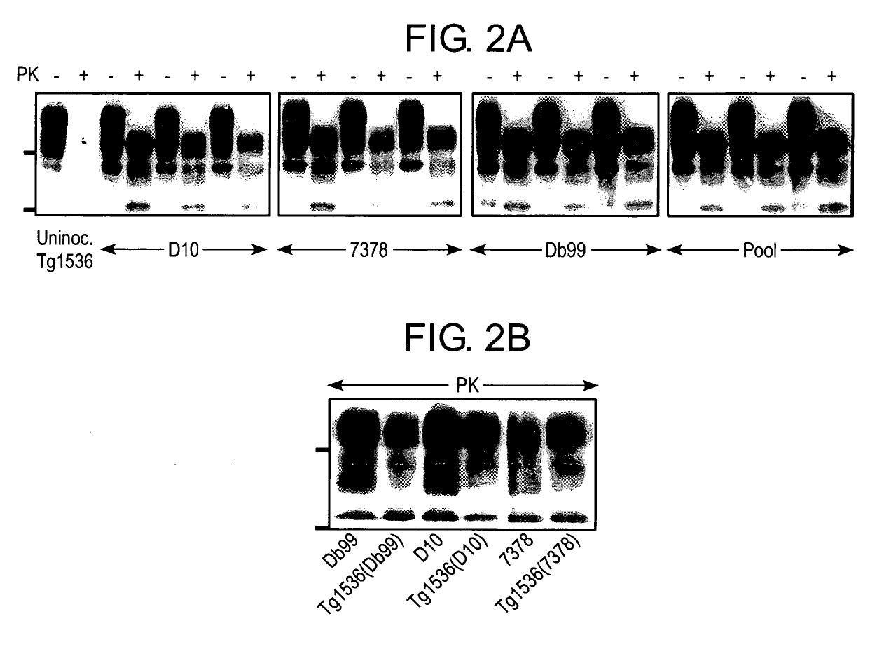

FIG. 2 shows Western blots of PrP in brains from Tg(CerPrP)1536^{+/-} mice inoculated with prions from mule deer and elk with CWD A. The brains of Tg(CerPrP)1536^{+/-} mice inoculated with D10, 7378, Db99 and CWD pool were analyzed for the presence of protease resistant PrP^{Sc}. Brain extracts of three individual brains from each inoculated group were treated (+) or not treated (−) with 40 μg/ml proteinase K (PK) in the presence of 2% sarkosyl for 1 hour at 37° C. In B, PrP^{Sc} in brain homogenates of Tg(CerPrP) 1536^{+/-} mice were directly compared with the corresponding CWD inocula from deer and elk. Immunoblots were probed with recombinant Fab Hum-P which recognizes an epitope on PrP between amino acid residues 96 to 105 (Safar, J. G. et al. (2002) Measuring prions causing bovine spongiform encephalopathy or chronic wasting disease by immunoassays and transgenic mice, Nat Biotechnol 20:1147-50). The positions of protein molecular weight markers at 28.7 and 21.3 kDa (from top to bottom) are shown to the left of the immunoblots.

Biochemical analysis of prion proteins in brain extracts from clinically sick Tg mice showed that protease-resistant PrP^{Sc} was present in all inoculated groups. The diglycosylated form of PrP^{Sc} predominated in the brains of sick Tg(CerPrP)1536^{+/-} mice (see FIG. 2A). A similar PrP^{Sc} glycosylation pattern has been observed in previous analyses of CWD-affected deer and elk (Race, R. IE. et al. (2002) Comparison of abnormal prion protein glycoform patterns from transmissible spongiform encephalopathy agent-infected deer, elk, sheep, and cattle, J Virol 76:12365-8. Comparison of PrP^{Sc} profiles in brain extracts of sick Tg(CerPrP)I536^{+/-} mice showed that the molecular weight and glycosylation pattern of PrP^{Sc} was consistent among all inoculated groups. However, while the amounts of diglycosylated and unglycosylated PrP^{Sc} in CWD-affected cervids and CWD-affected Tg(CerPrP)1536^{+/-} mice remained constant, the amount of monoglycosylated PrP^{Sc} was consistently lower following transmission of Db99, D10 and 7378 brain extracts to Tg(CerPrP)1536^{+/-} mice (FIG. 2B). Similar differences in glycoform ratios of the same prion strain propagated in mice and human brain have been observed previously (Hill, A. F. et al. (1997) The same prion strain causes vCID and BSE, Nature 389:448-450).

### Example 5

### Neuroanatomical Studies of CWD Infected Tg(CerPrP) Mice

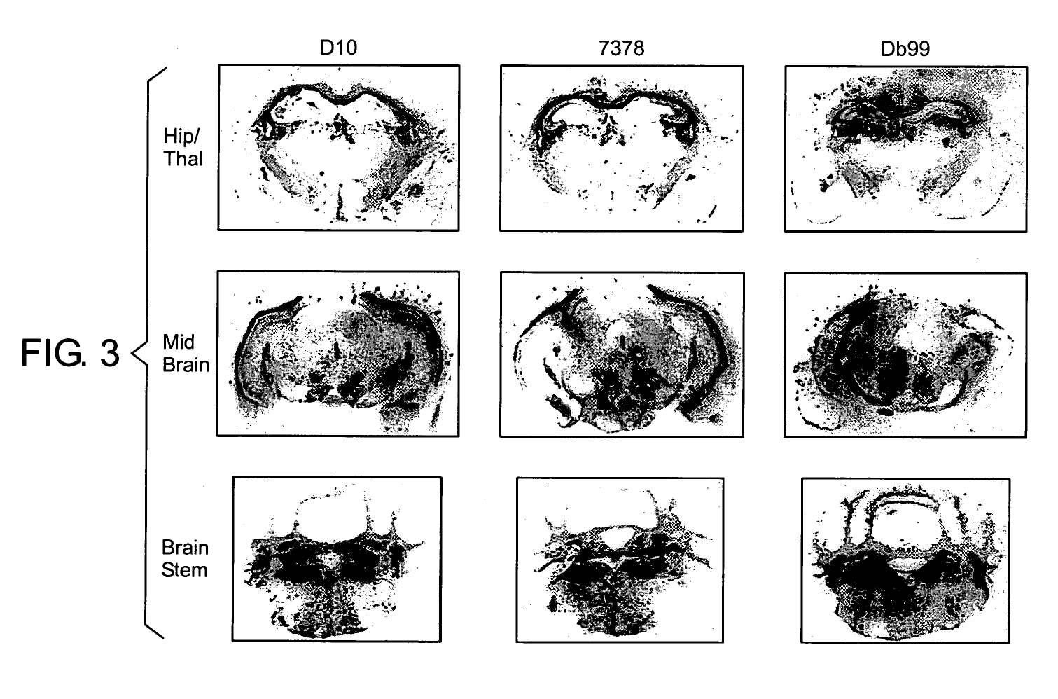

FIG. 3 shows regional distribution of PrP^{Sc} in the brains of Tg(CerPrP) I536^{+/-} mice inoculated with CWD prions. Histoblots of 10 μm thick cryostat sections were generated as previously described (Taraboulos, A. et al. (1992) Regional mapping of prion proteins in brains, Proc. Natl. Acad. Sci. USA 89:7620-7624). To eliminate PrP^{C} from the section, the membranes were air dried, rehydrated for 30 min. and exposed for 1 hour at 37° C. to 400 mg/ml proteinase K. To enhance immunostaining of PrP^{Sc}, the histoblots were exposed to antibody. Histoblotted coronal sections through the hippocampus and thalamus, the midbrain and the brain stem of Tg(CerPrP)1536^{+/-} mice inoculated with CWD mule deer isolates D10 and Db99 and CWD elk isolate 7378 are shown.

The neuroanatomical distribution of PrP^{Sc} was assessed by histoblotting as described previously (Taraboulos, A. et al. (1992) Regional mapping of prion proteins in brains, Proc. Natl. Acad. Sci. USA 89:7620-7624). The most notable feature of histoblotted Tg(CerPrP)1536^{+/-} mouse brains inoculated with CWD prions from D10, Db99 mule deer and 7378 elk was the widespread punctate deposition of PrP^{Sc} (see FIG. 3), which corresponds to the PrP^{Sc}-containing plaques detected by immunohistochemistry (see FIG. 1). The concordant patterns of PrP^{Sc} deposition in coronal sections of Tg(CerPrP)1536^{+/-} mice inoculated with prions from the D10 CWD-positive mule deer and the 7478 CWD-positive elk along with the similar incubation times, histopathologic findings and biochemical properties of PrP^{Sc} indicate that the same CWD prion strain caused disease in these analyzed mule deer and elk. Although the incubation time in Tg(CerPrP) 1536^{+/-} mice of the Db99 CWD mule deer isolate was similar to the D10/7378 strain, the difference in the neuroanatomical distribution of PrP^{Sc} in Db99-inoculated Tg(CerPrP) 1536^{+/-} mice (see FIG. 3) suggests that a different prion strain caused CWD in the Db99 infected mule deer.

### Production and Characterization of Transgenic Mice for CWD Transmission

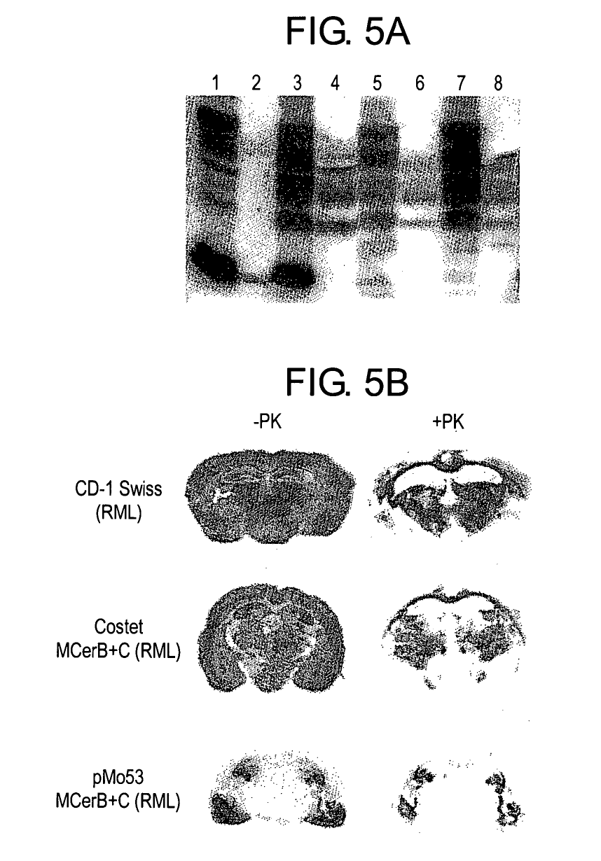

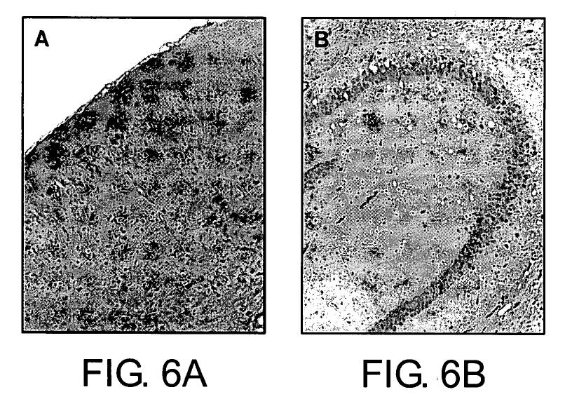

Tg mice expressing chimeric Cer/mouse PrP (MCerB+C) were produced using two different constructs: a plasmid-based construct, referred to as pMo53 and the cosmid construct cosSHa.tet. Microinjection of these constructs resulted in Tg(MCerB+C) lines 3 and 849, respectively. Transgene expression levels in Tg3 mice were ˜0.5-1.0 fold the level of PrP in CD-I Swiss mice, while MCerB+C expression levels in Tg849 mice are ˜2- to 4-fold higher than wild type (see FIG. 5A). Tg849 mice homozygous for the transgene express double the amount of transgene-encoded MCerB+C PrP. CWD prions from various sources inoculated into Tg3 mice failed to produce neurodegenerative disease. MCerB+C PrP expression was localized to the CA1/CA2 regions of the hippocampus and amygdala of the cerebrum (FIG. 5B) while regional PrP^{C} expression in the brains of Tg849 mice was similar to CD-1 Swiss mice. The highly restricted regional expression of transgene-encoded PrP^{C} in the brains of Tg3 mice was due to pMo53 lacking control elements for more global CNS expression, and that this lack of global expression might be the cause of inefficient CWD transmission in these mice. However, neither hemizygous or homozygous Tg849 mice inoculated with CWD material from a number of different sources developed disease, in some cases up to >500 days post inoculation. In contrast, and, based on the design of the expression cassette, paradoxically, Tg(MCerB+C) are highly susceptible to mouse-adapted RML scrapie prions with incubation times in low-expressing Tg3 mice of 386±5 (mean±SEM) (9/9 mice inoculated). Sub passage of material from scrapie sick Tg(MCerB+C) line 3 mice to CD1 Swiss mice resulted in a highly reduced incubation time of 117±3 days (9/9 mice inoculated). Sub passage into Tg(MCerB+C) mice is ongoing. The incubation time of mouse-adapted RML scrapie prions in Tg 894 mice was much shorter at 170±1.2 days (7/7 mice inoculated), the shorter incubation time presumably resulting from increased and more widespread expression of the transgene in the CNS. The brains of clinically sick Tg3 and Tg849 mice inoculated with RML prions contain PrP^{Sc} by Western blot and histoblot (see FIG. 5B); immunohistochemical and hisotpathological analysis of brains from RML-inoculated Tg3 mice also revealed PrP^{Sc} accumulation and spongiform degeneration of white matter (see FIG. 6).

CWD inoculation studies have been initiated in Tgi536^{+/-} mice which express Kozak-optimized CerPrP encoding methionine at residue 132 at levels up to 6 times higher than wild type PrP. No mice have become sick yet with the most advanced experiments being 117 days post inoculation of CWD material. Tg mice expressing Kozak-optimized CerPrP encoding leucine at residue 132 have also been produced: five founders were identified and three lines were bred for further analysis. Tg1970 and Tg1973 express CerPrP, L132 at levels ˜2-fold higher than wild type mice, while Tg1972 express CerPrP, L132 at levels ˜16-fold higher than wild type mice. Interestingly, Tg1972 mice appear to develop spontaneous neurological disease at ˜3 months of age. Tg1973 mice have recently been inoculated with CWD material.

FIG. 6A shows transgenic models of human, bovine and ovine prion diseases as a means of assessing susceptibility of humans and livestock to CWD infection.

New chimeric mouse/human PrP expression constructs were produced for more effective human prion transmission in Tg mice. MHu2M-VQ is a variant of MHu2M in which two additional type A residues are changed from human to mouse. Recently published studies demonstrate that Tg mice expressing this construct have abbreviated CJD prion incubation times compared to Tg(MHu2M) mice. Chimeric mouse-human PrP constructs were also produced in which only type A residues in the human PrP coding sequence are replaced with the corresponding mouse PrP residues, referred to as Hu2MHu and Hu2MHu-VQ. Tg mice are being produced expressing naturally occurring scrapie alleles from sheep. Studies of natural scrapie in sheep have confirmed the importance of three amino acid codons in the sheep PrP gene (136, 154 and 171) (Table 2). The ovine PrP alleles designed for transgenic mouse production are variants at amino acid residues 136, 154 and 171. The following alleles have been cloned into cosSHa.tet: valine 136, arginine 154, glutamine 171 (VRQ); alanine 136, histidine 154, glutamine 171 (AHQ); alanine 136, arginine 154, glutamine 171 (ARQ), and; alanine 136, arginine 154, arginine 171 (ARR). Transgenic expression constructs containing naturally occurring bovine PrP alleles with 5 or 6 octapeptide repeats were also produced.

TABLE 2
Suffolk and Cheviot sheep PrP genotypes and natural scrapie

a) Suffolk sheep:
PrP Genotype Natural scrapie
ARQ/ARQ High risk of scrapie
ARQ/ARR Occasional occurrence
ARR/ARR Resistant
b) Cheviot sheep:
PrP Genotype Natural scrapie
VRQ/VRQ Very high risk of scrapie
VRQ/ARQ Very high risk of scrapie
VRQ/ARR Occasional occurrence
ARQ/ARQ Resistant
ARQ/ARR Resistant
ARR/ARR Resistant

Microinjection of cosmid constructs has to date produced the following results:

TABLE 3
Ovine and Bovine PrP transgenic mouse production

| Transgene | Live births | Founders |
|---|---|---|
| AHQ | 15 | 6 |
| ARQ | 27 | 6 |
| VRQ | 18 | 4 |

Founders were bred and we are in the process of characterizing mice in the F1 generation.

### Development of Cervid PrP^{c} Transgenetic Mice to Study CWD Prion Infectivity, Transmission, Host Range, Pathogenesis, and Therapeutics

The production of transgenic (Tg) mice expressing high levels of transgene-encoded PrP with short CWD prion incubation times is a high priority. A new transgenic expression construct was developed, pMo53, and produced a number of Tg lines. While pMo53-derived transgenic mice express transgene-encoded PrP in the CNS, levels of expression are not as high as previously observed using cosSHa.Tet expression constructs. Two approaches were used to counteract this problem. CosSHa.Tet was obtained and additional transgenic expression constructs using this construct were produced. While the rate of transgenic founder production was considerably lower than with pMo53-derived constructs, lines were produced with higher levels of transgene-encoded PrP. An additional problem appeared to relate to inefficient translation of cervid PrP sequences resulting from a non-optimal Kozak consensus translation initiation sequence. Therefore, modified cervid (Cer) PrP constructs were produced with perfect Kozak initiation sequences. Additional transgenic lines have been produced expressing CerPrP and chimeric mouse/cerPrP with PrP expression driven by the cosSHa.Tet construct.

### Artificial Chimeric Mouse/Cervid PrP Expression Constructs

Two new Tg lines expressing chimeric Cer/mouse PrP (MCerB+C) have been produced. One line expressed at levels below wild type, the other line (Tg849) expresses 2×- to 4×-higher than wild type PrP and has been bred for transmission studies. Tg mice homozygous for the transgene were also produced and express double the amount of transgene-encoded MCerB+C PrP. Tg849^{+/+} mice have been inoculated with several CWD isolates. An additional Cer/mouse PrP chimera has been produced referred to as MCerPrP. This construct encodes a chimera in which the signal peptide derives from the mouse PrP sequence and CerPrP distal to this.

### Production of High Expressing Tg(CerPrP) Mice

Two new lines of Tg mice expressing Kozak-optimized CerPrP encoding methionine at residue 132 were produced. Tg1536 expresses ˜6×-higher than wild type PrP and Tg1534 express ˜2×-higher than wild type PrP. Lines are being bred for homozygosity of the transgene. Meanwhile, CWD inoculation studies have been initiated in TgI536^{+/-} mice. Tg mice expressing Kozak-optimized CerPrP encoding leucine at residue 132 have also been produced: five founders have been identified and these are being bred for further analysis.

### Characterization of Transgenic Mice

The original pMo53-derived MCerB+C mice (Tg3) were inoculated with CWD as well as mouse adapted scrapie RML prions. It was hypothesized that optimal CWD transmission would occur in Tg mice expressing this chimeric mouse/CerPrP construct containing type C and type B residues from CerPrP and type A residues derived from mouse PrP. It was expected that CerPrP type B and type C residues would facilitate efficient interactions between PrP^{Sc} in CWD inocula and transgene-encoded PrP^{C}, while the mouse type A residues would allow efficient interaction of transgene-encoded PrP^{C} with mouse protein X. Based on these hypotheses, it was surprising to discover that Tg3 mice were susceptible to RML mouse prions with an incubation period of 386±5 (n=9) and, so far, not CWD infectivity. Brains of clinically sick Tg3 mice inoculated with RML were analyzed and contain PrP^{Sc} and hisotpathological feature of prion disease. Serial transmission studies are currently ongoing.

Homozygous Tg22 and 26 express CerPrP encoding methionine at 132 at levels approximately equal to wild type PrP; homozygous Tg54 express CerPrP encoding leucine at 132 at levels approximately two fold higher than wild type PrP. Mice were inoculated with several CWD isolates but no evidence of prion transmission has been evident, in some cases after time periods exceeding 600 days.

### To Assess Selected Other Animal Species for Susceptibility to CWD Infection

Transgenic models of human, bovine and ovine prion diseases as a means of assessing susceptibility of humans and livestock to CWD infection were created. Chimeric mouse/human PrP expression constructs were created for more effective human prion transmission in Tg mice. MHu2M-VQ is a variant of MHu2M in which two additional type A residues are changed from human to mouse. Recently published studies demonstrate that Tg mice expressing this construct have abbreviated CJD prion incubation times compared to Tg(MHu2M) mice.

A chimeric mouse-human PrP construct was also produced in which only type A residues in the human PrP coding sequence are replaced with the corresponding mouse PrP residues, referred to as Hu2MHu and Hu2MHu-VQ. Tg mice expressing naturally occurring susceptible and non-susceptible scrapie alleles as well as naturally occurring bovine PrP alleles with 5 or 6 octapeptide can also be generated.

### Example 6

### Testing of Skeletal Muscle of Diseased Cervids for Prion Activity

Tg(CerPrP) mice expressing cervid prion protein (CerPrP) were inoculated intracerebrally with extracts prepared from the semitendinosus/semimembranosus muscle group of CWD-affected mule deer or from CWD-negative deer. The availability of CNS materials also allowed for direct comparisons of prion infectivity in skeletal muscle and brain. All skeletal muscle extracts from CWD-affected deer induced progressive neurological dysfunction in Tg(CerPrP) mice, with mean incubation times ranging between 360 and 490 days, whereas the incubation times of prions from the CNS ranged from 230 to 280 days. For each inoculation group, the diagnosis of prion disease was confirmed by the presence of disease-associated, protease-resistant PrP (PrPSc) in the brains of multiple infected Tg(CerPrP) mice. In contrast, skeletal muscle and brain material from CWD-negative deer failed to induce disease in Tg(CerPrP) mice, and PrPSc was not detected in the brains of asymptomatic mice as late as 523 days after inoculation.

Incubation Times After Inoculation of Tg(CerPrP) Mice with Prions from Skeletal Muscle and Brain Samples of CWD-Affected Deer. PBS, Phosphate Buffered Saline.

Incubation time, mean days ±
SEM (n/n0)*

| | | |
|---|---|---|
| Inocula | Skeletal muscle | Brain |

CWD-affected deer

| | | | | |
|---|---|---|---|---|
| H92 | 360 ± 2 | (6/6) | 283 ± 7 | (6/6) |
| 33968 | 367 ± 9 | (8/8) | 278 ± 11 | (6/6) |
| 5941 | 427 ± 18 | (7/7) | | |
| D10 | 483 ± 8 | (8/8) | 231 ± 17 | (7/7) |
| D08 | 492 ± 4 | (7/7) | | |

| | | |
|---|---|---|
| Averages | 426 | 264 |

Nondiseased deer

| | | | | |
|---|---|---|---|---|
| FPS 6.98 | >523 | (0/6) | | |
| FPS 9.98 | >454 | (0/7) | >454 | (0/6) |
| None | >490 | (0/6) | | |
| PBS | >589 | (0/5) | | |

*The number of mice developing prion disease (n) divided by the original number of inoculated mice (n0) is shown in parentheses. Mice dying of intercurrent illnesses were excluded.

The results show that skeletal muscle, as well as CNS tissue, of deer with CWD contains infectious prions. Similar analyses of skeletal muscle from BSE-affected cattle did not reveal high levels of prion infectivity.

Although PrPSc has been detected in muscles of scrapie-affected sheep, previous studies failed to detect PrPSc by immunohistochemical analysis of skeletal muscle from deer with natural or experimental CWD. Because the time of disease onset is inversely proportional to prion dose, the longer incubation times of prions from skeletal muscle extracts compared with those from matched brain samples indicated that prion titers were lower in muscle than in the CNS, where infectivity titers are known to reach high levels. Although possible effects of CWD strains or strain mixtures on these incubation times cannot be excluded, the variable 360-to-490-day incubation times suggested a range of prion titers in skeletal muscles of CWD-affected deer. Muscle prion titers at the high end of the range produced the fastest incubation times, which were 30% longer than the incubation times of prions from the CNS of the same animal. Because all mice in each inoculation group developed disease, prion titers in muscle samples producing the longest incubation times were higher than the end point of the bioassay, defined as the infectious dose at which half the inoculated mice develop disease.

Although the risk of exposure to CWD infectivity after consumption of prions in muscle is mitigated by relatively inefficient prion transmission via the oral route, the results show that semitendinosus/semimembranosus muscle, which is likely to be consumed by humans, is a major source of prion infectivity. See Science 24 Feb. 2006, Vol. 311. no. 5764, p. 1117

The information presented above is provided to give those of ordinary skill in the art with a complete disclosure and description of how to make and use the preferred embodiments of the invention, and is not intended to limit the scope of what the inventor(s) regard(s) as his or her/their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A non-human transgenic animal for studying chronic wasting disease (CWD), modified to express cervidprion protein (CerPrP), wherein said non-human transgenic animal produces PrP^{sc} upon infection with prions.

2. The non-human transgenic animal of claim 1, wherein the said non-human transgenic animal is selected from the group consisting of rodents, guinea pigs, rabbits, non-human primates, sheep, dogs, cows, amphibians, reptiles, avian such as meat bred and egg laying chicken and turkey, ovine such as lamb, bovine such as beef cattle and milk cows, piscine and porcine.

3. A method for making a non-human transgenic animal of claim 1, comprising the steps of:
a) constructing a construct containing an open reading frame of CerPrP; and
b) transforming the mouse with the construct.

4. A targeting construct comprising a coding sequence encoding CerPrP, the construct being suitable for genetic therapy.

5. (canceled)

6. (canceled)

7. A method for screening for therapeutic agents useful for treating prion associated disease, comprising:
a) inoculating a potential agent for treating prion-associated disease; and
b) determining the effects of the potential therapeutic agent on the development of prion-associated disease in the animal model of claim 1.

8. The method of claim 7, wherein the prion-associated disease is selected from the group consisting of Scrapie in sheep, TME (transmissible mink encephalopathy) in mink, CWD (chronic wasting disease) in muledeer and elk, BSE (bovine spongiform encephalopathy) in bovines and particularly cows, CJD (Creutzfeld-Jacob Disease) in humans, GSS (Gerstmann-Straussler-Scheinker syndrome) in humans, FFI (Fatal familial Insomnia) in humans, Kuru in humans, and Alpers Syndrome in humans.

9. A method for studying the molecular and biochemical events associated with prion disease, comprising:
a) inoculating transgenic CerPrP mice;
b) inoculating wild-type mice; and
c) comparing signs of prion disease from the mice in step a) to the mice in step b).

10. The method of claim 9, wherein the prion disease is selected from the group consisting of Scrapie in sheep, TME (transmissible mink encephalopathy) in mink, CWD (chronic wasting disease) in muledeer and elk, BSE (bovine spongiform encephalopathy) in bovines and particularly cows, CJD (Creutzfeld-Jacob Disease) in humans, GSS (Gerstmann-Straussler-Scheinker syndrome) in humans, FFI (Fatal familial Insomnia) in humans, Kuru in humans, and Alpers Syndrome in humans.
